(19) United States
(12) Patent Application Publication
(10) Pub. No.: US 2020/0147112 A1
(43) Pub. Date: May 14, 2020

(54) DIET THERAPY OF INTRACTABLE CANCER AND MEDICAL DIET SUITABLE THEREFOR

(71) Applicants: MEIJI CO., LTD., Tokyo (JP); THE NISSHIN OILLIO GROUP, LTD., Tokyo (JP); OSAKA UNIVERSITY, Osaka (JP)

(72) Inventors: Keisuke HAGIHARA, Osaka (JP); Noriyuki IKEDA, Kanagawa (JP); Kinya ASHIDA, Tokyo (JP); Kentaro NAKAMURA, Tokyo (JP)

(73) Assignees: MEIJI CO., LTD., Tokyo (JP); THE NISSHIN OILLIO GROUP, LTD., Tokyo (JP); OSAKA UNIVERSITY, Osaka (JP)

(21) Appl. No.: 16/587,551

(22) Filed: Sep. 30, 2019

(30) Foreign Application Priority Data
Oct. 4, 2018 (JP) 2018-188865

(51) Int. Cl. A61K 31/7016; A61K 35/00; A61K 45/06

(52) U.S. Cl. CPC A61K 31/7016; A61K 45/06; A61K 35/00

(57) ABSTRACT

An object of the present invention is to provide a diet therapy for suppressing tumor growth of refractory cancer, for treating refractory cancer, or for enhancing the effect of another cancer treatment. The present invention provides a saccharide restricted high fat composition for use in suppressing tumor growth of refractory cancer, treating refractory cancer and/or enhancing the effect of another cancer treatment. The composition of the present invention can comprise about 55% by mass or more of fat based on the total solid content contained in the composition. The composition of the present invention can also comprise about 0% by mass to about 15% by mass of saccharide based on the total solid content contained in the composition. The composition of the present invention can also comprise a medium chain fatty acid oil as fat.

### CROSS-REFERENCE TO RELATED APPLICATIONS

The present application enjoys the benefit of priority from the prior Japanese Patent Application No. 2018-188865 filed on Oct. 4, 2018, the entire disclosure of which is incorporated herein by reference.

### BACKGROUND OF THE INVENTION

### Technical Field

The present invention relates to a diet therapy for refractory cancer and a medical diet suitable therefor.

### Background Art

Recently, along with Westernization of eating habits, cancers often found in Western countries, such as colorectal cancer, breast cancer, lung cancer and prostate cancer, have increased instead of gastric cancer. In the case of carcinomas, such as gastric cancer and colorectal cancer, which are resectable upon early detection, patients' life prognoses are being improved. However, many of refractory cancers including pancreatic cancer and osteosarcoma are difficult to early detect, and clinical countermeasures exist as great problems. Existing cancer treatment mainly involves surgical resection, a chemotherapy and a radiation therapy, and the chemotherapy cannot be said to provide a sufficient therapeutic effect on lung cancer and pancreatic cancer. Accordingly, there exists a further demand for development of effective therapies for cancer patients, and, amongst others, a novel therapy for refractory cancer patients is strongly demanded.

Ketone body is a generic term for acetoacetic acid, β-hydroxybutyric acid and acetone, and is synthesized in the liver by β-oxidation of fatty acids in the living body. In humans, when glucose is insufficiently supplied due to fasting state or long-term exercise, fat is decomposed to produce ketone bodies from fatty acids, and the ketone bodies are used as an energy source (Vidali S, et al., Int J Biochem Cell Biol. 63, 55-59 (2015)). As a meal devised to produce many ketone bodies in the body, a ketogenic diet, which is a high-lipid low-saccharide diet, has been traditionally known. Upon intake of a ketogenic diet, the concentration of ketone bodies in the blood rises. A diet therapy using a ketogenic diet is known to be useful in treating refractory epilepsy and GLUTI deficiency and suppressing refractory epileptic seizure (Tatsuya Fujii, “From Basic to Practice of Ketogenic Diet,” SHINDAN TO CHIRYO SHA, Inc. (2011)).

### SUMMARY OF THE INVENTION

An object of the present invention is to provide a diet therapy for suppressing tumor growth of refractory cancer, for treating refractory cancer, or for enhancing the effect of another refractory cancer treatment, and a composition suitable for the diet therapy.

When carrying out a saccharide restricted high fat diet therapy on terminal cancer patients with pancreatic cancer, breast cancer, multiple bone metastasis, pharyngeal space malignancy, mammary gland malignancy, chondrosarcoma, astroblastoma, epipharyngeal cancer and abdominal wall liposarcoma to verify tumor growth, the present inventors have now found that tumor is reduced in size or that tumor growth is suppressed to be brought into a stable state. The present invention is based on these findings.

The present invention provides the following inventions.

[1] A saccharide restricted high fat composition for use in suppressing tumor growth of refractory cancer, treating refractory cancer and/or enhancing the effect of cancer treatment.

[2] The composition according to [1], which comprises about 55% by mass or more of fat based on the total solid content contained in the composition.

[3] The composition according to [1] or 121, which comprises a medium chain fatty acid oil as fat.

[4] The composition according to any one of [1] to [3], wherein about 30% by mass or more of fat is a medium chain fatty acid oil.

[5] The composition according to any one of [1] to [4], which comprises about 0% by mass to about 15% by mass of saccharide based on the total solid content contained in the composition.

[6] The composition according to any one of [1] to [5], which comprises lactose as saccharide.

[7] The composition according to any one of [1] to [6], which comprises about 5% by mass to about 40% by mass of protein based on the total solid content contained in the composition.

[8] The composition according to any one of [1] to [7], which comprises about 25% by mass to about 40% by mass of a long chain fatty acid oil, about 30% by mass to about 50% by mass of a medium chain fatty acid oil, about 0% by mass to about 15% by mass of saccharide, and about 10% by mass to about 30% by mass of protein based on the total solid content contained in the composition.

[9] The composition according to any one of [1] to [8], wherein a fat intake amount is 120 g or more per day based on a real body weight of 50 kg, or 70% or more of the total daily energy intake.

[10] The composition according to any one of [1] to [9], wherein a saccharide intake amount is 30 g or less per day based on a real body weight of 50 kg.

[11] The composition according to any one of [1] to [10], wherein the composition is fed so that daily caloric intake by the composition is 1000 kcal or more based on a real body weight of 50 kg.

[12] The composition according to any one of [1] to [11], wherein the composition constitutes a part or all of modified Atkins diet, and wherein the modified Atkins diet is:

(1) for the first week, provided to the subject at a daily calorie of about 1500 kcal and at a ratio of about 140 g of lipid:about 60 g of protein:about 10 g of saccharide (carbohydrate other than dietary fibers), based on a real body weight of 50 kg;

(2) from the second week to the third month, provided to the subject at a daily intake amount of saccharide of about 20 g or less, at a daily calorie of about 1400 to about 1600 kcal, and at a ratio of about 120 to about 140 g of lipid:about 70 g of protein:about 20 g of saccharide; and

(3) after the third month, provided to the subject at a daily intake amount of saccharide of about 30 g or less, at a single intake amount of saccharide of about 10 g/intake, and in accordance with (2) with respect to others.

[13] The composition according to any one of [1] to [12], wherein the refractory cancer is one or more selected from the group consisting of pancreatic cancer, breast cancer, multiple bone metastasis, pharyngeal space malignancy, mammary gland malignancy, chondrosarcoma, astroblastoma, epipharyngeal cancer and abdominal wall liposarcoma.

[14] The composition according to any one of [1] to [13] for use in combination with another cancer treatment.

[15] The composition according to [14], wherein the another cancer treatment is selected from the group consisting of a surgical therapy, a chemotherapy and a radiation therapy, and a combination of some or all thereof.

[16] The composition according to any one of [1] to [15], which is fed to a stage 4 refractory cancer patient.

[17] The composition according to any one of [1] to [16], which is fed to a refractory cancer patient with a performance status of 2 or less.

[18] The composition according to any one of [1] to [17], which is a composition for oral intake or oral administration.

[19] The composition according to any one of [1] to [18], which is provided in any form selected from the group consisting of a frozen food product, a dairy product, a chilled food product, a nutritional food product, a liquid food, a nursing food and a beverage.

[20] A saccharide restricted high fat diet for use in suppressing tumor growth of refractory cancer, treating refractory cancer and/or enhancing the effect of cancer treatment.

[21] The saccharide restricted high fat diet according to [20], wherein a fat intake amount is 120 g or more per day based on a real body weight of 50 kg, or 70% or more of the total daily energy intake.

[22] The saccharide restricted high fat diet according to [20] or [21], wherein a saccharide intake amount is 30 g or less per day based on a real body weight of 50 kg.

[23] The saccharide restricted high fat diet according to any one of [20] to [22], wherein daily caloric intake is 1000 kcal or more based on a real body weight of 50 kg.

[24] The saccharide restricted high fat diet according to any one of [20] to [23], wherein the high fat diet is modified Atkins diet, and wherein the modified Atkins diet is:

(1) for the first week, provided to the subject at a daily calorie of about 1500 kcal and at a ratio of about 140 g of lipid:about 60 g of protein:about 10 g of saccharide (carbohydrate other than dietary fibers), based on a real body weight of 50 kg;

(2) from the second week to the third month, provided to the subject at a daily intake amount of saccharide of about 20 g or less, at a daily calorie of about 1400 to about 1600 kcal, and at a ratio of about 120 to about 140 g of lipid:about 70 g of protein:about 20 g of saccharide; and

(3) after the third month, provided to the subject at a daily intake amount of saccharide of about 30 g or less, at a single intake amount saccharide of about 10 g/intake, and in accordance with (2) with respect to others.

[25] The saccharide restricted high fat diet according to any one of [20] to [24], wherein a part or all of the high fat diet is composed of the saccharide restricted high fat composition according to any one of [1] to [19].

[26] The saccharide restricted high fat diet according to any one of [20] to [25], wherein the high fat diet is composed of the saccharide restricted high fat composition according to any one of [1] to [19] and a medium chain fatty acid oil.

[27] A method for suppressing tumor growth of refractory cancer, a method for treating refractory cancer or a method for enhancing the effect of cancer treatment, comprising feeding or administering a saccharide restricted high fat composition to a subject in need thereof.

[28] The method according to [27], wherein the composition comprises about 55% by mass or more of fat based on the total solid content contained in the composition.

[29] The method according to [27] or [28], wherein the composition comprises a medium chain fatty acid oil as fat.

[30] The method according to any one of [27] to [29], wherein about 30% by mass or more of fat in the composition is a medium chain fatty acid oil.

[31] The method according to any one of [27] to [30], wherein the composition comprises about 0% by mass to about 15% by mass of saccharide based on the total solid content contained in the composition.

[32] The method according to any one of [27] to [31], wherein the composition comprises lactose as saccharide.

[33] The method according to any one of [27] to [32], wherein the composition comprises about 5% by mass to about 40% by mass of protein based on the total solid content contained in the composition.

[34] The method according to any one of [27] to [33], wherein the composition comprises about 25% by mass to about 40% by mass of a long chain fatty acid oil, about 30% by mass to about 50% by mass of a medium chain fatty acid oil, about 0% by mass to about 15% by mass of saccharide, and about 10% by mass to about 30% by mass of protein based on the total solid content contained in the composition.

[35] The method according to any one of [27] to [34], wherein a fat intake amount or fat dose is 120 g or more per day based on a real body weight of 50 kg, or 70% or more of the total daily energy intake.

[36] The method according to any one of [27] to [35], wherein a saccharide intake amount or fat dose is 30 g or less per day based on a real body weight of 50 kg.

[37] The method according to any one of [27] to [36], wherein the composition is fed or administered so that daily caloric intake by the composition is 1000 kcal or more based on a real body weight of 50 kg.

[38] The method according to any one of [27] to [37], wherein the composition constitutes a part or all of modified Atkins diet, and wherein the modified Atkins diet is:

(1) for the first week, provided to the subject at a daily calorie of about 1500 kcal and at a ratio of about 140 g of lipid:about 60 g of protein:about 10 g of saccharide (carbohydrate other than dietary fibers), based on a real body weight of 50 kg;

(2) from the second week to the third month, provided to the subject at a daily intake amount of saccharide of about 20 g or less, at a daily calorie of about 1400 to about 1600 kcal, and at a ratio of about 120 to about 140 g of lipid:about 70 g of protein:about 20 g of saccharide; and

(3) after the third month, provided to the subject at a daily intake amount of saccharide of about 30 g or less, at a single intake amount saccharide of about 10 g/intake, and in accordance with (2) with respect to others.

[39] The method according to any one of [27] to [38], wherein the refractory cancer is one or more selected from the group consisting of pancreatic cancer, breast cancer, multiple bone metastasis, pharyngeal space malignancy, mammary gland malignancy, chondrosarcoma, astroblastoma, epipharyngeal cancer and abdominal wall liposarcoma.

[40] The method according to any one of [27] to [39], which is carried out in combination with another cancer treatment.

[41] The method according to [40], wherein the another cancer treatment is selected from the group consisting of a surgical therapy, a chemotherapy and a radiation therapy, and a combination of some or all thereof.

[42] The method according to any one of [27] to [41], wherein the composition is fed or administered to a stage 4 refractory cancer patient.

[43] The method according to any one of [27] to [42], wherein the composition is fed or administered to a refractory cancer patient with a performance status of 2 or less.

[44] The method according to any one of [27] to [43], wherein the composition is a composition for oral intake or oral administration.

[45] The method according to any one of [27] to [44], wherein the composition is provided in any form selected from the group consisting of a frozen food product, a dairy product, a chilled food product, a nutritional food product, a liquid food, a nursing food and a beverage.

[46] A method for suppressing tumor growth of refractory cancer, a method for treating refractory cancer or a method for enhancing the effect of cancer treatment, comprising feeding or administering a saccharide restricted high fat diet to a subject in need thereof.

[47] The method according to [46], wherein a fat intake amount or fat dose is 120 g or more per day based on a real body weight of 50 kg, or 70% or more of the total daily energy intake.

[48] The method according to any one of [46] to [47], wherein a saccharide intake amount or saccharide dose is 30 g or less per day based on a real body weight of 50 kg.

[49] The method according to any one of [46] to [48], wherein daily caloric intake is 1000 kcal or more based on a real body weight of 50 kg.

[50] The method according to any one of [46] to [49], wherein the high fat diet is modified Atkins diet, and wherein the modified Atkins diet is:

(1) for the first week, provided to the subject at a daily calorie of about 1500 kcal and at a ratio of about 140 g of lipid:about 60 g of protein:about 10 g of saccharide (carbohydrate other than dietary fibers), based on a real body weight of 50 kg;

(2) from the second week to the third month, provided to the subject at a daily intake amount of saccharide of about 20 g or less, at a daily calorie of about 1400 to about 1600 kcal, and at a ratio of about 120 to about 140 g of lipid:about 70 g of protein:about 20 g of saccharide; and

(3) after the third month, provided to the subject at a daily intake amount of saccharide of about 30 g or less, at a single intake amount saccharide of about 10 g/intake, and in accordance with (2) with respect to others.

[51] The method according to any one of [46] to [50], wherein a part or all of the high fat diet is composed of the saccharide restricted high fat composition according to any one of [1] to [19].

[52] The method according to any one of [46] to [51], wherein the high fat diet is composed of the saccharide restricted high fat composition according to any one of [1] to [19] and a medium chain fatty acid oil.

Since the saccharide restricted high fat diet of the present invention can suppress tumor growth of refractory cancer for which no effective treatment method is considered to have been developed, the present invention provides a novel method for treating refractory cancer. Also, the present invention can be combined with normal methods for treating cancer such as a surgical therapy, a chemotherapy and a radiation therapy to reduce the number of applications and level of such normal cancer treatment methods, and thus is advantageous in contributing not only to improvement of patient QOL, but also to reduction of medical expenses.

### DETAILED DESCRIPTION OF THE INVENTION

### <<Saccharide Restricted High Fat Diet>>

The “saccharide restricted high fat diet” provided by the present invention means a high fat diet which is restricted in saccharide. Here, the “high fat diet” refers to a diet which allows intake of fat corresponding to about 30% or more of energy based on the total energy intake. This numerical value is based on the fact that 30% or more of the total energy intake is normally derived from lipid, according to the National Health and Nutrition Survey 2005 and 2006, 2) and 3). The lower limit value of the fat intake amount to be provided by the “high fat diet” can be set to about 40%, about 45%, about 50%, about 55%, about 60%, about 65%, about 70%, about 75%, about 80%, about 85% or about 90% based on the total energy intake, and the upper limit value thereof can be set to about 95%, about 90%, about 85% or about 80% based on the total energy intake. The lower limit value and upper limit value can be combined arbitrarily, and the “high fat diet” can allow intake of fat corresponding to about 50% to about 95%, about 60% to about 90%, about 65% to about 85% or about 70% to about 80% based on the total energy intake. It should be noted that the energy ratio is calculated on the premise that 1 g of fat produces 9 kcal. Also, the “high fat diet” refers to a diet which allows intake of fat in an amount of about 80 g or more per day based on a real body weight of 50 kg. The lower limit value of the fat intake amount to be provided by the “high fat diet” can be set to about 90 g, about 100 g, about 110 g, about 115 g, about 120 g, about 125 g, about 130 g, about 135 g, about 140 g, about 145 g or about 150 g, and the upper limit value thereof can be set to about 180 g, about 170 g, about 160 g, about 150 or about 140 g. The lower limit value and upper limit value can be combined arbitrarily, and the “high fat diet” can allow intake of fat in an amount of about 80 g to about 180 g per day, about 90 g to about 170 g per day, about 100 g to about 160 g per day, about 110 g to about 150 g per day, or about 120 g to about 140 g per day based on a real body weight of 50 kg.

Also, the “saccharide restricted” refers to feeding about 100 g or less of saccharide per day based on a real body weight of 50 kg. This numerical value is calculated based on the description that “Supposing that the basal metabolic rate is 1,500 kcal/day, the energy consumption of the brain is 300 kcal/day which corresponds to 75 g/day of glucose. Since tissues other than the brain also utilize glucose as an energy source as described above, the necessary amount of glucose is estimated to be at least 100 g/day, i.e., the minimum necessary amount of digestible carbohydrate is estimate to be approximately 100 g/day” in the nutrition report in 2010 by the Ministry of Health, Labour and Welfare. It is understood that this numerical value can vary. The upper limit value of the saccharide intake amount to be provided by the saccharide restricted diet can be set to about 90 g, about 80 g, about 70 g, about 60 g, about 50 g, about 40 g. about 35 g, about 30 g, about 25 g, about 20 g, about 15 g or about 10 g, and the lower limit value thereof can be set to about 5 g, about 10 g, about 15 g, about 20 g, about 25 g, about 30 g or about 60 g. The lower limit value and upper limit value can be combined arbitrarily, and the saccharide restricted diet can allow intake of saccharide in an amount of about 60 g to about 70 g per day, about 5 g to about 15 g per day, about 15 g to about 25 g per day, or about 25 g to about 35 g per day based on a real body weight of 50 kg.

In a preferred embodiment of the “saccharide restricted high fat diet” provided by the present invention, the intake amount of saccharide in the introduction period may be further restricted. For example, the intake amount of saccharide may be restricted to about 20 g/day or less or about 10 g/day or less. The intake amount of saccharide in the introduction period is further limited, thereby making it possible rapid induction of blood ketone bodies (acetoacetic acid and f-hydroxybutyric acid), resulting in improvement in prognosis of cancer patients. However, the contents of meals at the early stage of introduction are different from conventional eating habits, and thus are difficult to continue. Gradual relaxation of the restriction on the saccharide intake amount makes it possible to continue the saccharide restricted high fat diet, and a therapeutic effect is also observed. Accordingly, the restricted amount of saccharide (intake amount of carbohydrate) is not limited, for example, to about 10 g/day->about 20 g/day->about 30 g/day, so long as the restriction on saccharide is characterized by being gradually relaxed from a strict restrict in the initial amount (for example, about 10 g/day or less). Thus, the amount of saccharide to be initially introduced can be started at about 5 g/day to about 15 g/day or thereabouts (±about 5 g/day) in some cases. At the second stage, the amount of saccharide can be maintained at about 15 g/day to about 25 g/day or thereabouts (±about 5 g/day). At the last maintenance stage, the amount of saccharide can be maintained at about 25 g/day to about 35 g/day or thereabouts (±about 10 g/day).

The saccharide restricted high fat diet of the present invention may comprise a monosaccharide such as glucose (grape sugar), fructose (fruit sugar) or galactose; a disaccharide such as maltose (malt sugar), sucrose (cane sugar) or lactose (milk sugar); or a polysaccharide such as starch (amylose or amylopectin), glycogen or dextrin, or any combination thereof, under restriction on saccharide (for example, daily intake amount of saccharide as described above). The saccharide restricted high fat diet of the present invention may also be free of glucose or a polysaccharide based on glucose as a basic component. The saccharide restricted high fat diet of the present invention preferably comprises lactose (milk sugar), more preferably is free of glucose and comprises lactose (milk sugar) as saccharide, and particularly preferably substantially comprises lactose (milk sugar) alone as saccharide, under restriction on saccharide (for example, daily intake amount of saccharide as described above), from the viewpoint of the tumor reduction/shrinkage effect and the improvement of prognosis.

The saccharide restricted high fat diet of the present invention can be fed so that daily caloric intake is about 20 kcal/kg body weight or more (about 1000 kcal or more based on the standard body weight of 50 kg). Without wishing to be bound to the following theory, this is because the improvement of prognosis can be expected by avoiding low calorie (about 600 kcal/day based on the standard body weight of 50 kg). Accordingly, even caloric intake of 20 kcal/kg body weight/day or less, for example, caloric intake of about 14 kcal/kg body weight/day or more, about 16 kcal/kg body weight/day or more or about 18 kcal/kg body weight/day or more can be utilized in some cases. Alternatively, in the present invention, the caloric intake may be preferably about 22 kcal/kg body weight/day or more, about 24 kcal/kg body weight/day or more, about 26 kcal/kg body weight/day or more, about 28 kcal/kg body weight/day or more, or the like. In a typical example, caloric intake of about 30 kcal/kg body weight/day may be utilized.

In a preferred embodiment of the “saccharide restricted high fat diet” provided by the present invention, whether a diet is a saccharide restricted high fat diet or not can be determined based on the ketogenic ratio (lipid/(protein+saccharide)) (by mass). The case where the ketogenic ratio is equal to or higher than about 1 (lipid is contained in a large amount) is preferable as the saccharide restricted high fat diet. Any ratio may be employed when the ketogenic ratio is about 1 or more, and the ketogenic ratio may be set, for example, to about 1 to about 2, and is preferably about 2 at the time of introduction. Any amounts of protein and saccharide can be employed so long as the ketogenic ratio satisfies this definition. The amounts of protein and saccharide are preferably about 30 g or less per day, more preferably about 20 g or less per day, further preferably about 10 g or less per day, or may be a combination thereof depending on the period. The single intake amount may be within any range so long as it falls within the daily intake amount range, and can preferably be set to about 10 g or less.

In a more preferred embodiment of the “saccharide restricted high fat diet” provided by the present invention, Atkins diet may be used for children, and modified Atkins diet may be used for adults. The modified Atkins diet is as follows.

(1) For the first week, the goal is: a calorie of about 30 kcal/kg body weight, no restriction on lipid or protein, and about 10 g or less of saccharide (carbohydrate other than dietary fibers), based on a real body weight. Specifically, based on a real body weight of 50 kg at the early stage of introduction, the goal is: a daily caloric intake of about 1500 kcal and a ratio of about 140 g of lipid:about 60 g of protein:about 10 g of saccharide, and a ketogenic ratio (lipid/(protein+saccharide)) of 2. Other nutrients can be fed without restriction. Necessary trace elements and/or vitamins are appropriately fed through the use of supplements or the like. The period can appropriately be extended/shortened, and may be set to several days to several weeks.

(2) For the second week to the third month, the saccharide amount and the intake amount of medium chain fatty acids by a ketogenic formula and an MCT oil are adjusted with reference to the blood ketone body values. For example, guidance is given so that acetoacetic acid is not 500 μmol/L or more and β-hydroxybutyric acid is not 1000 μmol/L or less, and, if possible, the target values are set to 1000 μmol/L or more for acetoacetic acid and 2000 μmol/L or more for p-hydroxybutyric acid. The goal is: a daily intake amount of saccharide of about 20 g or less, a daily calorie of about 1400 to about 1600 kcal, a ratio of about 120 to about 140 g of lipid:about 70 g of protein:about 20 g of saccharide, and a ketogenic ratio of about 1 to about 2. At the time of calorie supply, an MCT oil and a ketogenic formula can preferably be used. The period may appropriately be extended/shortened, which may be slightly longer or shorter than two weeks, and the third month may also be slightly shifted ahead or behind (about one, two or several weeks of shift is acceptable).

(3) After the third month, the daily intake amount of carbohydrate (carbohydrate other than dietary fibers, corresponding to saccharide) is set to about 30 g or less when the single intake amount thereof is 10 g/intake, and others are in accordance with (2). Since this period comes after (2), this period itself would be shifted when the third month is shifted.

The saccharide restricted high fat diet of the present invention can be provided as a combination of various modules, and it is understood that the diet is provided not only as one nutritional composition, but also as a kit or combination of various components (individual foods or nutritional compositions).

### <<Saccharide Restricted High Fat Composition>>

The present invention provides a nutritional composition suitable for the saccharide restricted high fat diet of the present invention. Specifically, the present invention provides a saccharide restricted high fat composition comprising a high content of fat and a low content of saccharide. Such a saccharide restricted high fat composition can be used so as to satisfy the feeding forms (for example, fat intake amount, saccharide intake amount and daily caloric intake) required of the saccharide restricted high fat diet of the present invention.

Briefly, the saccharide restricted high fat composition of the present invention can comprise a high content of fat. The lower limit value of the content ratio of fat in the saccharide restricted high fat composition of the present invention can be set to about 55% by mass (preferably, about 60% by mass, about 70% by mass or about 80% by mass) based on the total solid content contained in the composition, and the upper limit value thereof can be set to about 95% by mass or about 90% by mass based on the total solid content contained in the composition. The lower limit value and upper limit value can be combined arbitrarily, and the range of the content ratio of fat based on the total solid content contained in the composition can be set, for example, to about 55% by mass to about 95% by mass or about 55% by mass to about 90% by mass.

The content ratio of fat in the saccharide restricted high fat composition of the present invention can be expressed by the ratio (% energy, which may be indicated as mere “%” herein) of the fat energy amount (kcal) based on the total energy amount (kcal) of the composition. The lower limit value of the ratio can be set to about 70% (preferably about 80%, about 85% or about 90%), and the upper limit value thereof can be set to about 95% or about 90%. The lower limit value and upper limit value can be combined arbitrarily, and the range of the content ratio of fat in the saccharide restricted high fat composition of the present invention (ratio of the fat energy amount based on the total energy amount of the composition) can be set, for example, to about 70% to about 95% or about 80% to about 90%.

In the present invention, fat can include a short chain fatty acid oil, a medium chain fatty acid oil and a long chain fatty acid oil, and any combination thereof. The fat contained in the saccharide restricted high fat composition of the present invention and the saccharide restricted high fat diet of the present invention preferably have a high content ratio of a medium chain fatty acid oil. For example, the lower limit value of the ratio of the medium chain fatty acid oil based on the total fat can be set to about 10% by mass, about 20% by mass, about 30% by mass, about 40% by mass, about 50% by mass, about 60% by mass, about 70% by mass or about 80% by mass, and the upper limit value thereof can be set to about 90% by mass, about 80% by mass, about 70% by mass or about 60% by mass. The lower limit value and upper limit value can be combined arbitrarily, and the range of the ratio of the medium chain fatty acid oil based on the total fat in the saccharide restricted high fat composition of the present invention and the saccharide restricted high fat diet of the present invention is, for example, about 10% by mass to about 90% by mass, about 20% by mass to about 80% by mass, about 30% by mass to about 80% by mass, or about 40% by mass to about 70% by mass, and can be preferably set to about 50% by mass to about 60% by mass.

As used herein, the “medium chain fatty acid oil” means oil in which constituent fatty acids of oil and fat have a medium chain length, which is also called MCT (Medium Chain Triglyceride) or medium chain fatty acid triglyceride, and refers to oil composed of fatty acids typically with 6 to 12 carbon atoms, preferably with 8 to 12 carbon atoms, or oil composed of fatty acids with 8 to 11 carbon atoms or fatty acids with 8 to 10 carbon atoms. Medium chain fatty acid oils are more readily digested and absorbed, and converted to energy than common oils. Examples of such medium chain fatty acids include hexanoic acid (caproic acid; C6), octanoic acid (caprylic acid; C8), nonanoic acid (pelargonic acid; C9), decanoic acid (capric acid; C10), and dodecanoic acid (lauric acid; C12).

Since medium chain fatty acid oils exist in oils and fats contained in plant bodies including Palmae plants such as coconut and palm fruit and dairy products such as cow's milk, medium chain fatty acid oils extracted (including rough extraction) or purified (including rough purification) from these oils and fats (preferably, vegetable oils and fats such as palm kernel oil) can be used as they are or as raw materials. Alternatively, products and commercial products by chemical synthesis methods may each be used as medium chain fatty acid oils. As the medium chain fatty acid oil, Nisshin MCT oil, Nisshin MCT powder and extra virgin coconut oil (all manufactured by The Nisshin OilliO Group, Ltd.) can be used.

The saccharide restricted high fat composition of the present invention can have a restricted saccharide content. The lower limit value of the content ratio of saccharide in the saccharide restricted high fat composition of the present invention can be set to about 0% by mass or about 5% by mass based on the total solid content contained in the composition, and the upper limit value thereof can be set to about 15% by mass or about 10% by mass based on the total solid content contained in the composition. The lower limit value and upper limit value can be combined arbitrarily, and the range of the content ratio of saccharide based on the total solid content contained in the composition can be set, for example, to about 0% by mass to about 15% by mass or about 5% by mass to about 10% by mass.

The “saccharide” as used herein refers to a carbohydrate which is not dietary fibers, and includes monosaccharides, disaccharides and polysaccharides. Examples of monosaccharides include glucose (grape sugar), fructose (fruit sugar) and galactose. Examples of disaccharides include maltose (malt sugar), sucrose (cane sugar) and lactose (milk sugar). Examples of polysaccharides include starches (amylose and amylopectin), glycogen and dextrin.

The saccharide restricted high fat composition of the present invention and the saccharide restricted high fat diet of the present invention are preferably those comprising lactose as saccharide, more preferably those free of glucose and comprising lactose as saccharide, most preferably those substantially comprising lactose alone as saccharide. When the saccharide restricted high fat composition of the present invention and the saccharide restricted high fat diet of the present invention comprise lactose, for example, about 10% by mass or more, about 20% by mass or more, about 30% by mass or more, about 40% by mass or more, about 50% by mass or more, about 60% by mass or more, about 70% by mass or more, about 80% by mass or more, or about 90% by mass or more of the entire saccharide can be composed of lactose. Preferably, the entire saccharide (100% by mass of the saccharide) can be composed of lactose.

The “carbohydrate” as used herein refers to an organic compound comprising a monosaccharide as a component. The “carbohydrate” as used in the context of the saccharide restricted high fat composition and high fat diet of the present invention refers to a carbohydrate other than dietary fibers, i.e., saccharide. The “carbohydrate” and “saccharide” are used interchangeably.

The saccharide restricted high fat composition of the present invention may also comprise protein. The lower limit value of the content ratio of protein in the saccharide restricted high fat composition of the present invention can be set to about 5% by mass or about 10% by mass based on the total solid content contained in the composition, and the upper limit value thereof can be set to about 400% by mass or about 20% by mass based on the total solid content contained in the composition. The lower limit value and upper limit value can be combined arbitrarily, and the range of the content ratio of protein based on the total solid content contained in the composition can be set, for example, to about 5% by mass to about 40% by mass or about 100% by mass to about 20% by mass.

Preferred embodiments of the saccharide restricted high fat composition of the present invention include a composition comprising about 25% by mass to about 40% by mass of a long chain fatty acid oil, about 30% by mass to about 50% by mass of a medium chain fatty acid oil, about 0% by mass to about 15% by mass of saccharide, and about 10% by mass to about 30% by mass of protein based on the total solid content contained in the composition.

Also, preferred embodiments of the saccharide restricted high fat composition of the present invention include a composition in which the ketogenic ratio (lipid/(protein+saccharide)) (by mass) is about 1 or more (preferably about 2 or more, more preferably about 2.5 or more), and the upper limit value of the ketogenic ratio can be, for example, about 4 or about 3.5.

Preferred embodiments of the saccharide restricted high fat composition of the present invention include Ketonformula (817-B) (manufactured by Meiji Co., Ltd.) and a composition comprising components equivalent to those of the ketogenic formula as well as their modified products (for example, a product obtained by further reducing saccharide and/or protein in Ketonformula (817-B) (for example, a product in which the amounts of the respective components in Ketonformula (817-B) are independently changed by ±about 5%, ±about 10%, ±about 15%, ±about 20% or ±about 25%)). The composition of Ketonformula (817-B) is as indicated in Tables 1 and 2 which will be given below.

The saccharide restricted high fat composition of the present invention can be used as the saccharide restricted high fat diet of the present invention (preferably, modified Atkins diet). In a preferred embodiment of the present invention, the saccharide restricted high fat composition of the present invention can constitute a part or all of the modified Atkins diet. More preferably, the saccharide restricted high fat composition of the present invention can constitute a part or all of the modified Atkins diet in combination with a medium chain fatty acid oil.

The saccharide restricted high fat composition of the present invention is orally fed or administered as will be described later, and thus can be provided as a food product composition. The “food product” as used herein refers to a natural product containing one or more nutrients and a processed product thereof, includes all beverages and foods, and includes, for example, lunch box forms and sets of meal forms provided at restaurants or as meal service. The “food product,” when used for mammals other than humans, can be used in the meaning including feeds. Examples of food products include, but are not limited to, frozen food products, dairy products, chilled food products, nutritional food products, liquid foods, nursing foods and beverages. In another embodiment, the saccharide restricted high fat composition of the present invention is provided by gastrostomy in some cases. This is because it is impossible or difficult for terminal cancer patients to orally take food products in some cases. The saccharide restricted high fat composition of the present invention may be prepared by adding a medium chain fatty acid oil and/or a ketogenic formula. The saccharide restricted high fat composition of the present invention is a composition in which the nutrient composition is adjusted in advance, and thus is advantageous in implementing the saccharide restricted high fat diet of the present invention.

### <<Intended Use>>

The saccharide restricted high fat composition of the present invention and the saccharide restricted high fat diet of the present invention can be used in any or all of: suppressing tumor growth of refractory cancer, treating refractory cancer and enhancing the effect of another cancer treatment. Accordingly, the saccharide restricted high fat composition of the present invention can be used as a pharmaceutical product (for example, a pharmaceutical composition), and the saccharide restricted high fat diet of the present invention can be used as a medical diet.

Refractory cancer includes cancers of types and stages conventionally regarded as being difficult to treat, such as stage 4 (stage IV) terminal cancer, hardly resectable cancer, cancer difficult to early detect and metastatic cancer, and examples thereof include pancreatic cancer (including metastatic), breast cancer (including metastatic), multiple bone metastasis, pharyngeal space malignancy, mammary gland malignancy (including metastatic), chondrosarcoma, astroblastoma, epipharyngeal cancer and abdominal wall liposarcoma.

As used herein, the “metastasis” refers to a process in which cancer spreads or moves from a primary site in the body to any other region so that a similar cancerous lesion develops at a new place. The “metastatic” or “metastasizing” cells are cells which lose their adhesive contact with adjacent cells, move from a primary site of a disease through blood stream or lymph, and enter a neighborhood somatic structure.

The “suppressing tumor growth” is used herein in the meaning including not only reduction of tumor size, but also apparent termination of tumor growth (i.e., the tumor size is maintained). Also, the “treatment” is used herein in the meaning including alleviation and remission of a symptom as well as decrease in the rate of disease progression.

The saccharide restricted high fat composition of the present invention and the saccharide restricted high fat diet of the present invention are preferably used in combination of another cancer treatment. Any cancer treatment can be utilized as the cancer treatment so long as it is used as cancer treatment, and examples thereof include a surgical therapy (for example, resection/extraction), a chemotherapy and a radiation therapy. The saccharide restricted high fat composition of the present invention and the saccharide restricted high fat diet of the present invention are used in combination of another cancer therapy, thereby making it possible to enhance the effect of cancer treatment. That is, the saccharide restricted high fat composition of the present invention and the saccharide restricted high fat diet of the present invention can be used to assist existing cancer treatment, and can additionally be applied to refractory cancer patients who are under such cancer treatment.

The surgical therapy refers to resection of a cancer lesion, and, in case of metastasis to the surrounding tissue or lymph node of the organ, excision thereof with the lesion or extraction of the organ itself. If resection is possible, the surgical therapy is positively used even for early stage cancer or somewhat advanced cancer. A method that minimizes a region to be resected as much as possible (reduction surgery), laparoscopic surgery using an endoscope (miniature camera), thoracoscopic surgery and the like have also been developed, which are encompassed in the surgical therapy.

The chemotherapy is a treatment method that mainly kills cancer cells or suppresses the proliferation thereof with an anticancer agent. The method of administering an anticancer agent includes drip, injection, and oral administration. An anticancer agent systemically circulates through blood, and thus has an effect also on very small metastasis. Hormonal therapy (endocrine therapy) is also sometimes encompassed in this chemotherapy.

The radiation therapy is a local therapy involving irradiating a cancer lesion part with radiation to kill cancer cells. With the advancement in the pre-treatment inspection techniques and irradiation methods, it is possible to accurately measure the size and position of cancer and to localize the irradiation only to the cancerous portion. The radiation therapy includes not only “external irradiation” involving applying radiation from the outside of the body, but also “brachytherapy” involving inserting a needle or capsule with a radiation-emitting substance sealed therein into a lesion part, and “radionuclide therapy” involving administering a radioactive substance through injection or oral administration. X-rays are often used as the radiation used in the radiation therapy, but proton beam therapy or heavy ion beam (carbon ion beam) therapy using particle beams can also be used.

In the present invention, patients in any conditions can be targeted, but patients preferably with a performance status (PS) of 2 or less from the viewpoint of implementation of the diet therapy without no difficulty. Since diabetes patients require separate restriction on saccharide, target patients in the present invention can be those having no complication of diabetes. However, cancer treatment using the saccharide restricted high fat composition of the present invention and the saccharide restricted high fat diet of the present invention can be used in combination, by appropriately restricting saccharide so as to be adapted to diabetes as well. The reason why diabetes patients were excluded in an open trial test as will be described in the Examples below is because priority was given to safety at the time of submission to the Ethics Committee. Since it has been able to be confirmed that hypoglycemia does not occur due to supply of saccharide from muscles and the liver even under restriction on saccharide, the present invention can be carried out also on diabetes patients.

Examples of subjects to which the saccharide restricted high fat composition of the present invention and the saccharide restricted high fat diet of the present invention can be fed can include any mammals, including rodents such as mice, rats, hamsters, and guinea pigs; Lagomorpha such as rabbits; ungulates such as pigs, cows, goats, horses, and sheep; Carnivora such as dogs and cats; and primates such as humans, monkeys, rhesus monkeys, cynomolgus monkeys, marmosets, orangutans, and chimpanzees. Mammals are preferably rodents (mice or the like) or primates (humans or the like), more preferably primates, and still more preferably humans.

The saccharide restricted high fat composition of the present invention and the saccharide restricted high fat diet of the present invention can be orally fed or administered, but may sometimes be fed or administrated by drip, gastrostomy or the like. Thus, the saccharide restricted high fat composition of the present invention and the saccharide restricted high fat diet of the present invention can be provided in the form of infusion.

It is understood that the saccharide restricted high fat composition of the present invention and the saccharide restricted high fat diet of the present invention are normally fed in three divided doses per day, but may be fed in two divided doses or in a single dose per day, or conversely may be fed in four or five divided doses per day. As described above, separate formulations are provided in the introduction period (normally, the first week), the transition period (normally, the second week to the third month) and maintenance period (normally, after the third month). Therefore, the feeding or administering method may be appropriately changed or identical with each other.

### <<Diet Therapy>>

The diet therapy of the present invention is a method for suppressing tumor growth of refractory cancer, treating refractory cancer and/or enhancing the effect of cancer treatment, and can be carried out by providing the saccharide restricted high fat diet of the present invention to a subject. Specifically, the present invention provides a method for suppressing tumor growth of refractory cancer, a method for treating refractory cancer and a method for enhancing the effect of cancer treatment, comprising feeding the saccharide restricted high fat diet of the present invention to a subject in need thereof, as well as a method for suppressing tumor growth of refractory cancer, a method for treating refractory cancer and a method for enhancing the effect of cancer treatment, comprising feeding the saccharide restricted high fat composition of the present invention to a subject in need thereof, and these methods are encompassed in the diet therapy of the present invention.

The diet therapy of the present invention (i.e., the method for suppressing tumor growth of refractory cancer, the method for treating refractory cancer and the method for enhancing the effect of cancer treatment according to the present invention) can comprise the following steps of:

(1) for the first week (the period may appropriately be extended or shortened, which may be several days to several weeks), providing a meal to the subject at a calorie of about 30±10 kcal/kg body weight, under no restriction on lipid or protein, and at an amount of saccharide (carbohydrate other than dietary fibers) of about 40 g or less, desirably 10 g or less, based on a real body weight (preferably, providing a meal to the subject at a daily calorie of about 1500±500 kcal and at a ratio of about 110 to 170 g of lipid:about 35 to 85 g of protein:about 10 to 40 g of saccharide (ketogenic ratio (lipid/(protein+saccharide) is about 2±1), based on a real body weight of 50 kg, wherein other nutrients can be fed without restriction, and necessary trace elements and/or vitamins are appropriately fed through the use of supplements or the like);

(2) from about second week to about third month (the period may appropriately be extended or shortened, second week may be slightly shifted ahead or behind, and the third month may also be slightly shifted ahead or behind (about one, two or several weeks of shift is acceptable)), providing a meal to the subject at a daily intake amount of saccharide of about 40 g or less, at a daily calorie of about 1200 to about 2000 kcal, at a ratio of about 90 to 170 g of lipid:about 45 to 95 g of protein:about 10 to 40 g of carbohydrate, and at a target ketogenic ratio of about 1 to about 2, wherein, in some cases, guidance is given so that acetoacetic acid is not about 500 t 100 μmol/L or more and β-hydroxybutyric acid is not about 1000±200 mol/L or less with reference to the blood ketone body values, and, if possible, the target values are set to about 1000±200 μmol/L or more for acetoacetic acid about 2000±400 μmol/L or more for β-hydroxybutyric acid (in the above step, preferably providing a meal using a medium chain fatty acid oil and the saccharide restricted high fat composition of the present invention, more preferably providing a meal to the subject using a medium chain fatty acid oil and Ketonformula (817-B) or a modified product thereof); and

(3) after the third month (when the end of (2) is shifted, this period itself would be shifted), providing a meal to the subject at a daily intake amount of saccharide of about 75 g or less, desirably 30 g or less, at a single intake amount of saccharide of about 30 g/intake or less, and in accordance with (2) with respect to others.

The diet therapy of the present invention (i.e., the method for suppressing tumor growth of refractory cancer, the method for treating refractory cancer and the method for enhancing the effect of cancer treatment according to the present invention) can be carried out in accordance with the modified Atkins method, and can comprise the following steps:

(1) for the first week (the period may appropriately be extended or shortened, which may be several days to several weeks), providing a meal to the subject at a calorie of about 30 kcal/kg of body weight, under no restriction on lipid or protein, and at an amount of saccharide (carbohydrate other than dietary fibers) of about 10 g or less, based on a real body weight (preferably, providing a meal to the subject at a daily calorie of about 1500 kcal and at a ratio of about 140 g of lipid:about 60 g of protein:about 10 g of saccharide (ketogenic ratio (lipid/(protein+saccharide) is about 2), based on a real body weight of 50 kg, wherein other nutrients can be fed without restriction, and necessary trace elements and/or vitamins are appropriately fed through the use of supplements or the like);

(2) from about second week to about third month (the period may appropriately be extended or shortened, second week may be slightly shifted ahead or behind, and the third month may also be slightly shifted ahead or behind (about one, two or several weeks of shift is acceptable)), providing a meal to the subject at a daily intake amount of saccharide of about 20 g or less, at a daily calorie of about 1400 to about 1600 kcal, at a ratio of about 120 to about 140 g of lipid:about 70 g of protein:about 20 g of carbohydrate, and at a target ketogenic ratio of about 1 to about 2, wherein, in some cases, guidance is given so that acetoacetic acid is not about 500 μmol/L or more and β-hydroxybutyric acid is not about 1000 μmol/L or less with reference to the blood ketone body values, and, if possible, the target values are set to about 1000 μmol/L or more for acetoacetic acid and about 2000 μmol/L or more for β-hydroxybutyric acid (in the above step, preferably providing a meal using a medium chain fatty acid oil and the saccharide restricted high fat composition of the present invention, more preferably providing a meal to the subject using a medium chain fatty acid oil and Ketonformula (817-B) or a modified product thereof); and

(3) after the third month (when the end of (2) is shifted, this period itself would be shifted), providing a meal to the subject at a daily intake amount of saccharide of about 30 g or less, at a single intake amount of saccharide of about 10 g/intake, and in accordance with (2) with respect to others.

According to another aspect of the present invention, there is provided a composition for use in suppressing tumor growth of refractory cancer, treating refractory cancer and/or enhancing the effect of another refractory cancer treatment, comprising a medium chain fatty acid oil. According to a preferred embodiment of this composition, there is provided a composition comprising about 30% by mass or more, about 40% by mass or more, about 50% by mass or more, about 60% by mass or more, about 70% by mass or more or about 80% by mass or more of a medium chain fatty acid oil based on the total solid content contained in the composition. More preferably, a composition comprising about 90% by mass or more of a medium chain fatty acid oil based on the total solid content contained in the composition is provided.

The descriptions given herein are applied when the saccharide restricted high fat composition of the present invention and the saccharide restricted high fat diet of the present invention are used both for non-therapeutic purposes and for therapeutic purposes, and, in the case of therapeutic purposes, feeding can be read as administration.

The “mass” as used herein means a value in terms of solid content, unless otherwise specified. The “solid content” as used herein means a component except moisture.

The number of grams of the daily intake amount of fat, saccharide or protein described herein is based on a real body weight of 50 kg. The “real body weight” as used herein refers to the actual body weight Those skilled in the art understand that the number of grams of the daily intake amount can vary depending on the real body weight. For example, it is understood that the intake amount, when the real body weight is 80 kg, is 1.6 times the intake amount based on a real body weight of 50 kg.

According to another aspect of the present invention, there are/is provided an agent for suppressing tumor growth of refractory cancer, an agent for treating refractory cancer and/or an agent for enhancing the effect of cancer treatment, comprising a saccharide restricted high fat composition.

According to another aspect of the present invention, there is provided use of a saccharide restricted high fat composition, for the manufacture of an agent for suppressing tumor growth of refractory cancer, an agent for treating refractory cancer and/or an agent for enhancing the effect of cancer treatment, as an agent for suppressing tumor growth of refractory cancer, an agent for treating refractory cancer and/or an agent for enhancing the effect of cancer treatment, or for suppressing tumor growth of refractory cancer, treating refractory cancer and/or enhancing the effect of cancer treatment.

According to another aspect of the present invention, there is provided use of a saccharide restricted high fat diet, for the manufacture of an agent for suppressing tumor growth of refractory cancer, an agent for treating refractory cancer and/or an agent for enhancing the effect of cancer treatment, as an agent for suppressing tumor growth of refractory cancer, an agent for treating refractory cancer and/or an agent for enhancing the effect of cancer treatment, or for suppressing tumor growth of refractory cancer, treating refractory cancer and/or enhancing the effect of cancer treatment.

The agents and uses according to the present invention can be implemented in accordance with the descriptions concerning the saccharide restricted high fat composition of the present invention and the saccharide restricted high fat diet of the present invention.

As used herein, the phrase “X to Y” indicating a range means “X or more and Y or less.” Further, a numerical value to which the term “about” is added indicates significant figure(s) thereof, unless otherwise specified. A numerical value indicated as a “lower limit value” encompasses both the case of including values “not less than” the numerical value and the case of including values “more than” the numerical value. A numerical value indicated as an “upper limit value” encompasses both the case of including values “not more than” the numerical value and the case of including values “less than” the numerical value.

### EXAMPLES

Hereinafter, the present invention will be described in more detail by way of the following examples, but is not limited thereto. Clinical trials were conducted in compliance with the Declaration of Helsinki and the standards of the Osaka University.

### Example 1: Refractory Cancer Suppressing Effect of Ketogenic Diet

In this Example, a single-center open trial test was conducted on 13 cancer patients.

### (1) Selection of Subjects

### A. Criteria for Adaptation

Target patients are stage 4 cancer patients with a PS of 2 or less, who have been pathologically diagnosed by specimen resection or biopsy. The patients and their family members have agreed to continuation of the diet therapy.

### B. Criteria for Exclusion

Cancer patients who are incapable of oral intake or cancer patients with a PS of 3 or more are excluded from targets. Patients with a complication of diabetes are also excluded.

### C. Item to be Evaluated

For the selected patients, images by PET-CT before and after introduction of a ketogenic diet were evaluated. Specifically, an image before the beginning of feeding of the ketogenic diet and an image 3 months after the beginning of feeding of the ketogenic diet were evaluated.

### (2) Test Method

In addition to normal treatment (a surgical therapy, a chemotherapy or a radiation therapy), a ketogenic diet therapy was carried out on patients with various cancers. Specifically, modified Atkins diet was guided as the ketogenic diet. Explanation was given that temporary hypoglycemia, nausea, fatigue, or the like would appear at the time of introduction of the ketogenic diet. Actual nutritional guidance was given under the guidance of a nutritionist who had given the guidance of a ketogenic diet to epilepsy patients for a long time. Persons who would cook the ketogenic diet also attended the nutritional guidance. The details of the ketogenic diet therapy were as follows.

A. For the first week, the goal was: a calorie of about 30 kcal/kg body weight, no restriction on lipid or protein, and a daily intake amount of carbohydrate (carbohydrate other than dietary fibers, corresponding to saccharide; the same applies hereinafter) of about 10 g or less, based on the real body weight. Specifically, the goal was: a daily caloric intake of about 1500 kcal and a ratio of 140 g of lipid:60 g of protein: 10 g of saccharide, based on a real body weight of 50 kg, and a ketogenic ratio [lipid (g)/(protein (g)+saccharide (g))] of 2. Other nutrients could be fed without restriction. Necessary trace elements and/or vitamins were appropriately fed through the use of supplements or the like. At the time of introduction of the ketogenic diet, meals in accordance with the menu prepared by a nutritionist were fed.

B. For the second week to the third month, the saccharide amount and the intake amount of medium chain fatty acids by a ketogenic formula and an MCT oil were adjusted with reference to the blood ketone body values. Concerning the blood ketone body values, guidance was given so that acetoacetic acid was not 500 μmol/L or more and β-hydroxybutyric acid was not 1000 μmol/L or less, and, if possible, the target values were set to 1000 μmol/L, or more for acetoacetic acid and 2000 μmol/L or more for β-hydroxybutyric acid. The goal was: a daily intake amount of carbohydrate of 20 g or less, a daily caloric intake of 1400 to 1600 kcal, a ratio of 120 to 140 g of lipid:70 g of protein: 20 g of carbohydrate. At the time of calorie supply, a medium chain fatty acid oil (MCT oil, manufactured by The Nisshin OilliO Group, Ltd.) and Ketonformula (817-B) (manufactured by Meiji Co., Ltd.) were used. The composition of Ketonformula (817-B) was as indicated in Table 1.

TABLE 1
Composition (1) of Ketonformula (817-B)

| | In 100 g of | |
|---|---|---|
| Composition | Ketonformula (% E) | |
| Protein | 15.0 g | (8.1) |
| Lipid | 71.8 g | (87.2) |
| Carbohydrate | 8.8 g | (4.7) |
| Ash content | 2.4 g | (0) |
| Moisture | 2.0 g | (0) |
| Energy | 741 | kcal |

<Remarks>
Protein: milk protein
Lipid: long chain fatty acid oil and fat (essential fatty acid-adjusted fat) 32.1 g (39.0% E)
medium chain fatty acid oil and fat 39.7 g (48.2% E)
Carbohydrate: lactose
% E: % energy

Ketonformula (817-B) comprises, in addition to the main components indicated above, vitamin A, vitamin B1, vitamin B2, vitamin B6, vitamin B12, vitamin C, vitamin D, vitamin E, vitamin K, pantothenic acid, niacin, folic acid, calcium, magnesium, sodium, potassium, phosphorus, chlorine, iron, copper and zinc, and the composition thereof is as follows.

TABLE 2
Composition (2) of Ketonformula (817-B)

| Composition | In 100 g of Ketonformula | |
|---|---|---|
| Vitamin A | 600 | μg RE |
| Vitamin B₁ | 0.6 | mg |
| Vitamin B₂ | 0.9 | mg |
| Vitamin B₆ | 0.3 | mg |
| Vitamin B₁₂ | 4 | μg |
| Vitamin C | 50 | mg |
| Vitamin D | 12.5 | μg |
| Vitamin E | 6 | mg α-TE |
| Vitamin K | 30 | μg |
| Pantothenic acid | 2 | mg |
| Niacin | 6 | mg |
| Folic Acid | 0.2 | mg |
| Calcium | 350 | mg |
| Magnesium | 36 | mg |
| Sodium | 165 | mg |
| Potassium | 470 | mg |
| Phosphorus | 240 | mg |
| Chlorine | 320 | mg |
| Iron | 6 | mg |
| Copper | 350 | μg |
| Zinc | 2.6 | mg |

C. After the third month, the daily intake amount of carbohydrate was set to 30 g or less when the single intake amount thereof was 10 g/intake, and others were in accordance with (2).

The ketogenic diet (containing 75 to 89% of lipid) has been administered to child epileptic patients for a long term, so that the safety thereof has been confirmed. Thus, the diet is described in the 2010 edition of the COCHRANE LIBRARY. The document was also referred to as a reference for the actual policy. While nausea, fatigue, hypoglycemia, or the like may be temporarily observed at the early stage of introduction, it has been confirmed that they can be sufficiently dealt with. Since this is a high lipid diet, there is a possibility that a certain percentage of patients cannot continue the ketogenic diet due to their preference. It is possible to deal with such issues in cooperation with the nutritionist.

### (3) Evaluation Method

For the therapeutic effect on cancer, images by PET-CT before and after introduction of the ketogenic diet were determined in accordance with the RECIST guideline (Therasse P. et al., J Natl Cancer Inst, 2000, Vol 92, No. 3, 205-216).

### (4) Results

The results were as indicated in Table 3. The case where tumor was smaller by 30% or more (“partial response”) is indicated as PR, and the case where the tumor size was unchanged (“stable disease”) is indicated as SD.

TABLE 3
Therapeutic effect of ketogenic diet on cancer

| | | | | | | | | Period of |
|---|---|---|---|---|---|---|---|---|
| | | | Height | | | | Determination | continuation |
| Case | Sex | Age | (cm) | BMI | Disease | Stage | on treatment | (day) |
| 1 | F | 36 | 162 | 21.4 | Left parapharyngeal space | 4c | SD | 905 |
| | | | | | malignancy | | | |
| 2 | F | 41 | 167 | 20.1 | Mammary gland malignancy & | 4 | SD | 910 |
| | | | | | metastatic liver tumor | | | |
| 3 | M | 79 | 160.2 | 30.1 | Chondrosarcoma | 4 | SD | 882 |
| 4 | M | 44 | 172 | 18.3 | Anaplastic astroblastoma | 4 | SD | 735 |
| 5 | M | 55 | 167.7 | 22.3 | Epipharyngeal cancer | 4c | SD | 713 |
| 6 | F | 43 | 153.5 | 16.3 | Abdominal wall liposarcoma | 2B-3 | SD | 630 |
| 7 | F | 76 | 151 | 19.5 | Pancreatic tail cancer & multiple | 4 | PR | 406 |
| | | | | | liver metastases | | | |
| 8 | F | 74 | 143 | 22.3 | Pancreatic body cancer & lung | 4 | SD | 244 |
| | | | | | metastasis | | | |
| 9 | F | 69 | 156 | 19.4 | Re-recurrence after right breast | 4 | SD | 1250 |
| | | | | | cancer surgery | | | |
| 10 | F | 49 | 160 | 19.9 | Right breast cancer & liver | 4 | SD | 423 |
| | | | | | metastasis (suspicion of bone | | | |
| | | | | | metastasis) | | | |
| 11 | F | 54 | 148 | 22.0 | Multiple bone metastasis after | 4 | PR | 371 |
| | | | | | metachronous bilateral breast | | | |
| | | | | | cancer surgery | | | |
| 12 | F | 49 | 158 | 26.3 | Metastatic breast cancer | 4 | SD | 273 |
| 13 | F | 62 | 152.8 | 22.8 | Pancreatic body cancer, liver | 4 | SD | 518 |
| | | | | | metastasis & bone metastasis | | | |

*The period of continuation represents the number of days from the beginning of feeding of the ketogenic diet.

From the results in Table 3, the ketogenic diet therapy was observed to provide “partial response” in one case of pancreatic cancer and to provide “stable disease” in two cases thereof, and to provide “stable disease” in three cases of breast cancer. The diet was observed to provide “partial response” in one case of multiple bone metastasis, and to provide “stable disease” in one case of pharyngeal space malignancy, mammary gland malignancy, chondrosarcoma, astroblastoma, epipharyngeal cancer and abdominal wall liposarcoma, respectively. From the above results, the diet therapy of the preset invention was confirmed to suppress tumor growth of refractory carcinomas and to have a therapeutic effect thereon.

1. A method for suppressing tumor growth of refractory cancer, a method for treating refractory cancer or a method for enhancing the effect of cancer treatment, comprising feeding or administering a saccharide restricted high fat composition to a subject in need thereof.

2. The method according to claim 1, wherein the composition comprises about 55% by mass or more of fat based on the total solid content contained in the composition.

3. The method according to claim 1, wherein the composition comprises a medium chain fatty acid oil as fat.

4. The method according to claim 1, wherein about 30% by mass or more of fat in the composition is a medium chain fatty acid oil.

5. The method according to claim 1, wherein the composition comprises about 0% by mass to about 15% by mass of saccharide based on the total solid content contained in the composition.

6. The method according to claim 1, wherein the composition comprises lactose as saccharide.

7. The method according to claim 1, wherein the composition comprises about 5% by mass to about 40% by mass of protein based on the total solid content contained in the composition.

8. The method according to claim 1, wherein the composition comprises about 25% by mass to about 40% by mass of a long chain fatty acid oil, about 30% by mass to about 50% by mass of a medium chain fatty acid oil, about 0% by mass to about 15% by mass of saccharide, and about 10% by mass to about 30% by mass of protein based on the total solid content contained in the composition.

9. The method according to claim 1, wherein a fat intake amount or fat dose is 120 g or more per day based on a real body weight of 50 kg, or 70% or more of the total daily energy intake.

10. The method according to claim 1, wherein a saccharide intake amount or saccharide dose is 30 g or less per day based on a real body weight of 50 kg.

11. The method according to claim 1, wherein the composition is fed or administered so that daily caloric intake by the composition is 1000 kcal or more based on a real body weight of 50 kg.

12. The method according to claim 1, wherein the composition constitutes a part or all of modified Atkins diet, and wherein the modified Atkins diet is:
(1) for the first week, provided to the subject at a daily calorie of about 1500 kcal and at a ratio of about 140 g of lipid:about 60 g of protein:about 10 g of saccharide (carbohydrate other than dietary fibers), based on a real body weight of 50 kg;
(2) from the second week to the third month, provided to the subject at a daily intake amount of saccharide of about 20 g or less, at a daily calorie of about 1400 to about 1600 kcal, and at a ratio of about 120 to about 140 g of lipid:about 70 g of protein:about 20 g of saccharide; and
(3) after the third month, provided to the subject at a daily intake amount of saccharide of about 30 g or less, at a single intake amount of saccharide of about 10 g/intake, and in accordance with (2) with respect to others.

13. The method according to claim 1, wherein the refractory cancer is one or more selected from the group consisting of pancreatic cancer, breast cancer, multiple bone metastasis, pharyngeal space malignancy, mammary gland malignancy, chondrosarcoma, astroblastoma, epipharyngeal cancer and abdominal wall liposarcoma.

14. The method according to claim 1, which is carried out in combination with another cancer treatment.

15. The method according to claim 14, wherein the another cancer treatment is selected from the group consisting of a surgical therapy, a chemotherapy and a radiation therapy, and a combination of some or all thereof.

16. The method according to claim 1, wherein the composition is fed or administered to a stage 4 refractory cancer patient.

17. The method according to claim 1, wherein the composition is fed or administered to a refractory cancer patient with a performance status of 2 or less.

18. The method according to claim 1, wherein the composition is a composition for oral intake or oral administration.

19. The method according to claim 1, wherein the composition is provided in any form selected from the group consisting of a frozen food product, a dairy product, a chilled food product, a nutritional food product, a liquid food, a nursing food and a beverage.

20. A method for suppressing tumor growth of refractory cancer, a method for treating refractory cancer or a method for enhancing the effect of cancer treatment, comprising feeding or administering a saccharide restricted high fat diet to a subject in need thereof.

21. The method according to claim 20, wherein a fat intake amount or fat dose is 120 g or more per day based on a real body weight of 50 kg, or 70% or more of the total daily energy intake.

22. The method according to claim 20, wherein a saccharide intake amount or saccharide dose is 30 g or less per day based on a real body weight of 50 kg.

23. The method according to claim 20, wherein daily caloric intake is 1000 kcal or more based on a real body weight of 50 kg.

24. The method according to claim 20, wherein the high fat diet is modified Atkins diet, and wherein the modified Atkins diet is:
(1) for the first week, provided to the subject at a daily calorie of about 1500 kcal and at a ratio of about 140 g of lipid:about 60 g of protein:about 10 g of saccharide (carbohydrate other than dietary fibers), based on a real body weight of 50 kg;
(2) from the second week to the third month, provided to the subject at a daily intake amount of saccharide of about 20 g or less, at a daily calorie of about 1400 to about 1600 kcal, and at a ratio of about 120 to about 140 g of lipid:about 70 g of protein:about 20 g of saccharide; and
(3) after the third month, provided to the subject at a daily intake amount of saccharide of about 30 g or less, at a single intake amount of saccharide of about 10 g/intake, and in accordance with (2) with respect to others.
